(19) United States
(12) Patent Application Publication
(10) Pub. No.: US 2012/0076849 A1
(43) Pub. Date: Mar. 29, 2012

(54) PLANT COMPOSITION FOR THE TREATMENT OR PREVENTION OF VIRAL BLOOD-BORNE DISEASES SUCH AS DISEASES CAUSED BY THE HUMAN IMMUNODEFICIENCY VIRUS (HIV) OR HEPATITIS C

(75) Inventor: Sleimen El Kettany, Zouk Mikael (FR)

(73) Assignees: Nadia EL KHATIB, Beyrouth (LB); Sleimen EL KETTANY, Louveciennes (FR)

(21) Appl. No.: 13/263,810

(22) PCT Filed: Apr. 10, 2009
(86) PCT No.: PCT/FR2009/050678

§ 371 Date: Nov. 21, 2011

(51) Int. Cl. A61K 9/00; A61P 31/14; A61P 31/18; A61K 33/04; A61P 31/12

(52) U.S. Cl. 424/439; 424/705; 424/400

(57) ABSTRACT

A composition based on plants for treating or preventing viral diseases of the blood, such as the diseases caused by the virus of human immunodeficiency (HIV) or hepatitis C. The composition comprises flower of sulfur, a plant containing catechin and a tannin agent, and a pharmaceutically acceptable carrier. The plant is selected from Agrimonia Eupatoria and Uncaria gambir.

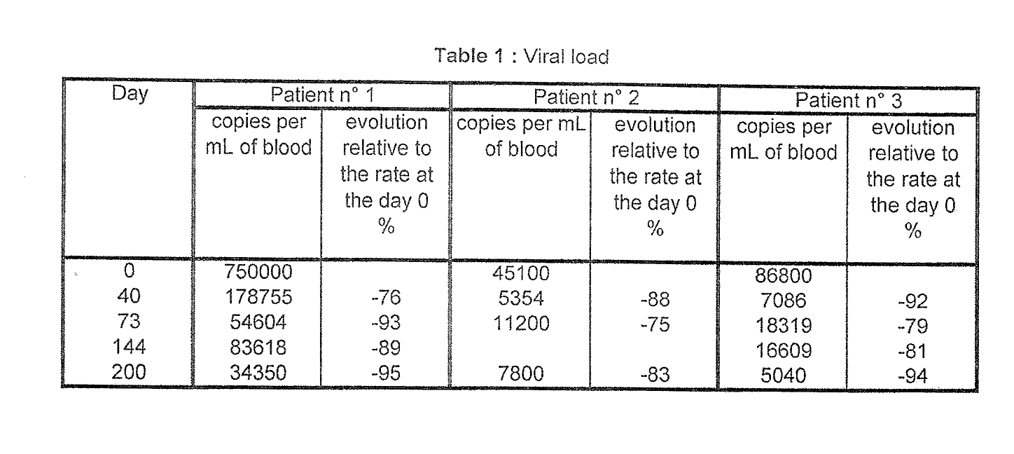

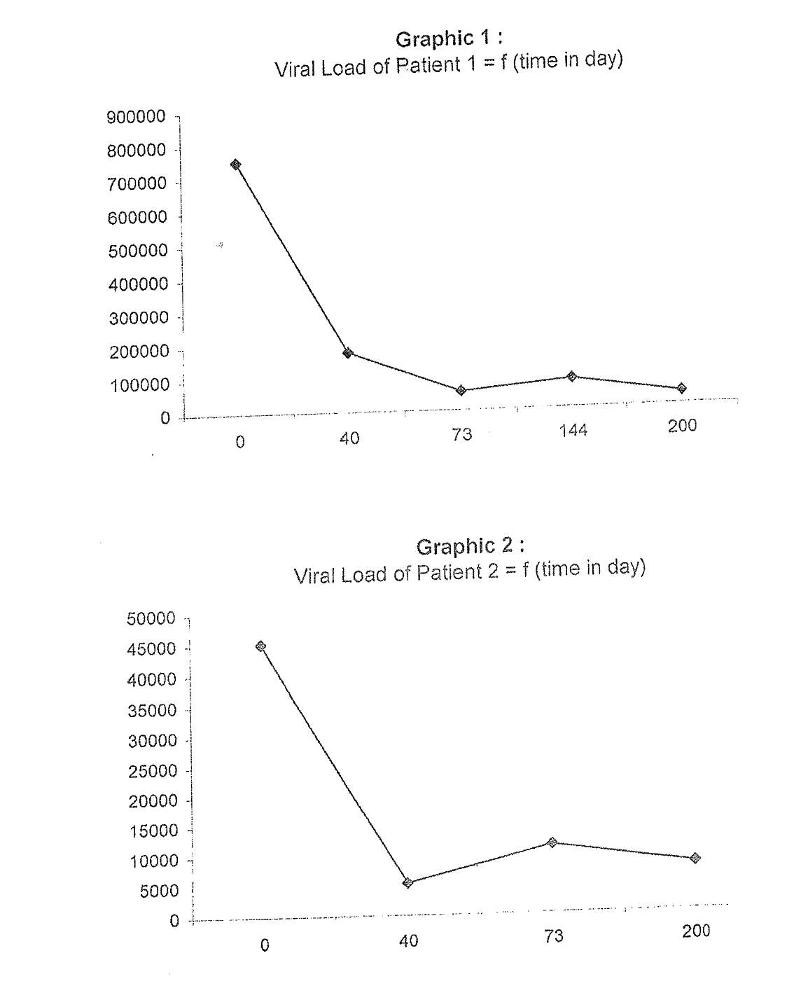

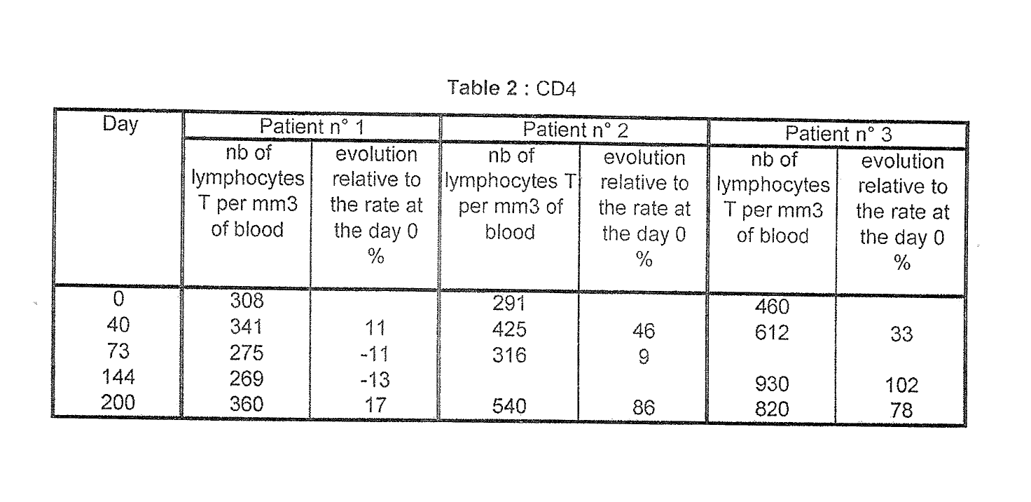

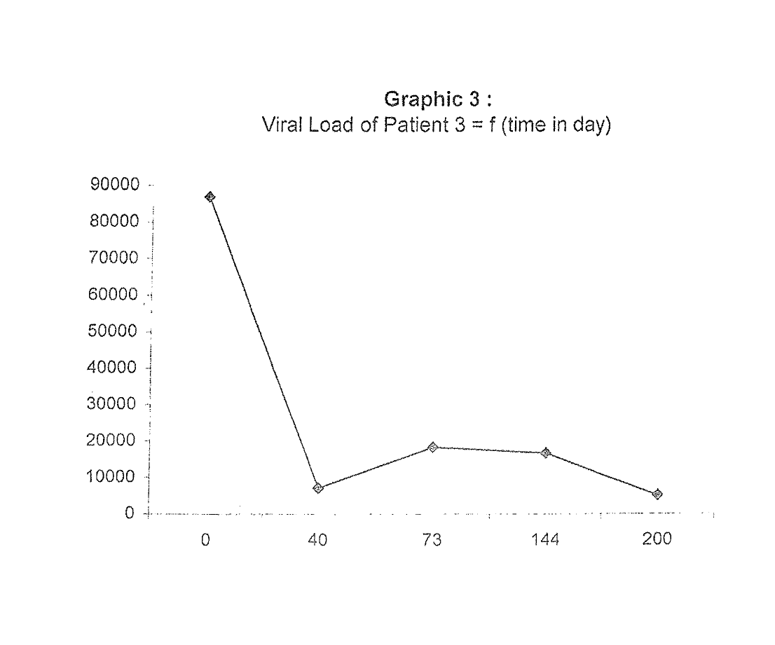

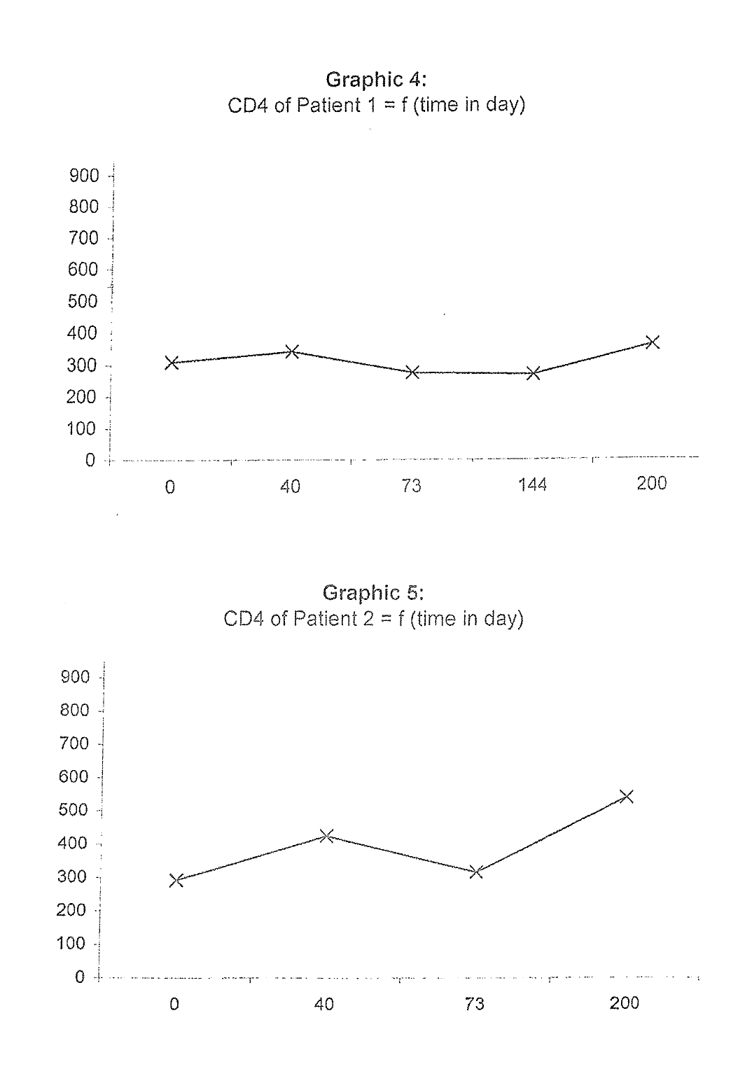

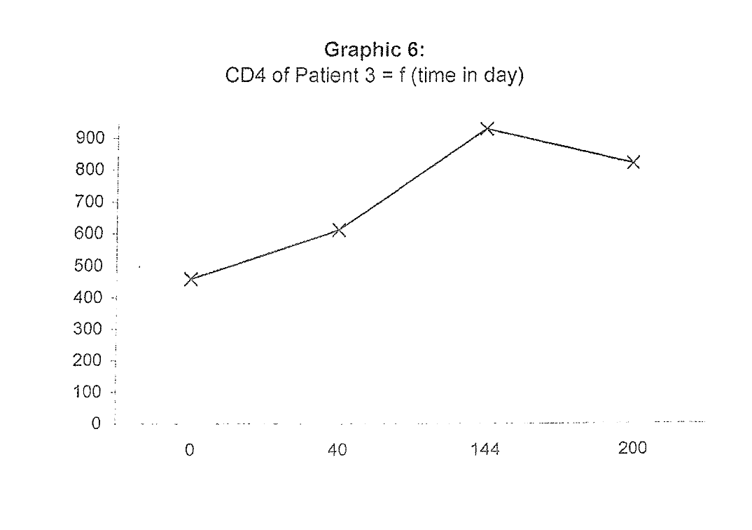

### FIELD OF THE INVENTION

The invention relates to a composition based on plants for treating or preventing viral blood diseases, such as diseases caused by the virus of human immunodeficiency (HIV) or hepatitis C.

HIV carriers belatedly develop symptoms related to the disease since the latter only arise after two to ten years following their contamination. During this period of time, as no effect of the disease is visible, the carriers may unknowingly transmit the virus during unprotected sexual intercourse, by blood contamination or by transmission from mother to child. This long incubation period is then partly responsible for an epidemic expansion of the disease.

Further, today, there is no vaccine providing protection against the HIV and the available known treatments such as retroviral tritherapies, have a certain efficiency but do not provide a cure.

Further, they are generally not prescribed at the beginning of seropositivity because they have many undesirable secondary effects, as well as a certain toxicity.

Among the most frequently and the less severe encountered side effects, are included headaches, nauseas and vomiting, fatigue, loss of appetite, bouts of fever, smarting or burning sensations on the hands and feet, diarrheas and skin problems.

Many side effects are persistent and become increasingly invalidating because of their duration. When the aforementioned passing effects persist, they may become irreversible and very crippling.

Other serious side effects only appear over time; they generally relate to the toxicity of the molecules.

And certain side effects are deadly:

cutaneous or respiratory hypersensitivities due to abacavir (Ziagen®);

cutaneous hypersensitivities and hepatic intolerance due to nevirapine (Viramune®);

acute pancreatitises due to ddI (Videx®).

Known conventional treatments therefore have not insignificant drawbacks.

### SUMMARY OF THE INVENTION

The invention proposes to solve this problem and the object thereof is a composition for treating or preventing viral blood diseases, effective in terms of reducing the viral load of a patient and increasing the number of T lymphocytes of the CD₄ subpopulation, and which generates less secondary effects in patients having ingested it or even none at all.

For this purpose, the invention relates to a composition based on plants for treating or preventing viral blood diseases such as diseases caused by the virus of human immunodeficiency (HIV) or hepatitis C.

According to the invention, the composition comprises flower of sulfur, at least one plant containing tannin agents and catechin, as well as a pharmaceutically acceptable carrier.

According to another feature, the plant containing catechin and tannin agents is selected from Agrimonia Eupatoria (GAFT) or the gambier tree (Uncaria gambir).

According to still another feature, the composition comprises two different plants containing tannin agents and catechin.

In this case, these plants are Agrimonia Eupatoria (GAFT) and the gambier tree (Uncaria gambir).

Advantageously, the composition further comprises Nigella.

More specifically, Nigella is present as an oil.

Further, the composition may comprise an antiputrid agent.

Preferably, the antiputrid agent is menthol.

According to another feature, the composition comprises a coating agent.

This coating agent is more particularly honey.

### BRIEF DESCRIPTION OF DRAWING FIGURES

The composition according to the invention as defined above thus exclusively derives from natural materials, oil of plants and of minerals extracted from the ground, mixed in predetermined proportions. It was proved by experimental tests that this product does not have any secondary effect of any kind and does not contain any stimulating, sedative or tranquilizing agent.

The invention also relates to the method for making the above composition.

This method comprises a step for mixing at least one plant containing tannin agents and catechin, with flower of sulfur and a pharmaceutically acceptable carrier.

More specifically, the plant containing catechin and tannin agents is selected from Agrimonia Eupatoria (GAFT) or the gambier tree (Uncaria gambir).

According to an alternative of interest, two different plants containing tannin agents and catechin are used, these plants being Agrimonia Eupatoria (GAFT) and the gambier tree (Uncaria gambir).

Advantageously, this method comprises a step for adding Nigella oil. Preferably, Agrimonia Eupatoria (GAFT) is used in the form of juice obtained by distillation.

Gambier is preferentially used as a finely ground powder.

According to another feature, the method comprises a preliminary step for dissolving menthol crystals in pure alcohol.

Preferably, the pharmaceutically acceptable support comprises honey with substantial viscosity.

This method is simple to apply since it mainly comprises a step for mixing Agrimonia Eupatoria (GAFT) juice, flower of sulfur, Nigella oil, gambier, with a pharmaceutically acceptable carrier which is honey.

### BRIEF DESCRIPTION OF DRAWING FIGURES

The invention will be better understood and other objects, advantages and features thereof will become apparent upon reading the following description, made with reference to the appended figures wherein:

Table 1 reports the viral load values measured on three patients to which the composition according to the invention was administered,

Graph 1 illustrates the change in the viral load on the first patient, to which the composition according to the invention was administered, versus time,

Graph 2 illustrates the change in the viral load on the second patient, to which the composition according to the invention was administered, versus time,

Graph 3 illustrates the change in the viral load on the third patient, to which the composition according to the invention was administered, versus time,

Table 2 reports the (CD4) T lymphocyte values measured over a period of 200 days in the three aforementioned patients,

Graphs 4-6 illustrate the change in the CD4 number versus time for the three patients respectively.

### DETAILED DESCRIPTION

The invention will be presented in a first part by a list of the main ingredients which make it up, in a second part by the method for making it from these ingredients, subsequently by its mode of administration to a patient and by its effectiveness as revealed by confirmed clinical tests carried out on patients. Finally, the detail of each ingredient is specified in a last part.

### Constitutive Ingredients

The composition according to the invention comprises the ingredients listed in the table below, and ideally in the proportions which are mentioned therein:

| | | | Range of mass | |
|---|---|---|---|---|
| | | Mass percent in | percentages capable | |
| | Mass in the | the composition | of ensuring | |
| | tested | tested on | effectiveness of the | Function(s) of the |
| | composition | patients | composition | ingredient considered |
| Ingredients: | (g) | (mass %) | (mass %) | individually |
| GAFT (latin name: | 5 | 0.54 | 0.54 = X □ 1.05 | two forms of catechins + |
| Agrimonia Eupatoria, | | | | and − , + catechin has |
| English name: | | | | an antibiotic effect and a |
| common agrimony) | | | | preventive action |
| | | | | against formation of |
| | | | | free radicals |
| Uncaria Gambir, | 4 | 0;43 | 0.43 < X < 1 | anti-inflammatory, anti- |
| English name: | | | | hemorrhagic |
| gambier tree | | | | |
| Nigella Sativa oil | 5 | 0.54 | 0.54 = X □ 0.87 | Analgesic, antiseptic, |
| (latin name: Nigella | | | | expectorant, antiseptic |
| Sativa L., English | | | | (against Gram-positive |
| name: nigella oil or | | | | and Gram-negative |
| black cumin seed oil) | | | | bacteria or organisms), |
| | | | | anti-colic, histamine |
| | | | | inhibitor, acts as a |
| | | | | bronchodilator |
| Edible flower of | 2 | 0.22 | 0.19 □ X □ 0.27 | energetic action at the |
| sulfur (or sublimed | | | | level of the liver |
| sulfur) | | | | |
| Menthol crystals | 1.5 | 0.16 | 0.15 □ X □ 0.18 | antiputrid |
| Pure alcohol (95° | 4 | 0.43 | 0.38 □ X □ 0.49 | solvent (of menthol and |
| ethanol) | | | | Nigella Sativa oils) |
| Pure honey | 900 | 97.67 | 92.24 □ X □ 99.83 | coats the composition, |
| | | | | antioxidant effect, |
| | | | | facilitates absorption by |
| | | | | the stomach |
| Total | 921.50 | 100.00 | | |

### Preparation Method

These ingredients are associated according to the following method:

1.5 gram of MENTHOL-CRYSTAL are placed inside a container, at room temperature and ambient pressure.

4 grams of pure alcohol, preferably 95° ethanol, are introduced into the container in order to dissolve the menthol crystals and, when the latter has been added, Nigella oil.

The whole is placed at rest for 5 minutes, and then stirred for assisting with the dissolution.

2 grams of edible flower of sulfur are incorporated into the obtained mixture.

5 grams of GAFT juice are then added. This juice is prepared beforehand by using 1 kg of dried leaves and stems of GAFT, soaked in 3.5 liters of water at room temperature for 20 hours. The mixture then undergoes a distillation process by being heated at a low temperature and by collecting the vapor formed in a cold container in order to collect about 2 liters of GAFT oil or GAFT juice.

The alcohol, the menthol, the flower of sulfur, and the GAFT juice are then mixed with 900 grams of pure honey, with substantial viscosity in order to facilitate absorption at the stomach.

4 grams of UNCARIA GAMBIR are finely ground and incorporated into the mixture.

5 grams of cold-extracted nigella oil (NIGELLA SATIVA OIL or HABAT EL BARAKAT), are incorporated into the mixture and dissolved in the latter.

### Mode of Administration

### Orally

About 20 grams of composition every 6 hours, four times daily, for at least 12 months, the effects of the composition on the body being observed as soon as 40 days after the beginning of the treatment.

Effectiveness within the Scope of Treating HIV-Affected Patients First Patient

In a first phase, the composition was used on a HIV-affected patient having a gastric ulcer in 2005, this patient being a woman of Cuban origin and of about 60 years of age. At that time, she was in a phase of the disease where all the symptoms of the latter were developing and she was further suffering from a severe gastric ulcer.

The composition was given to her with the goal of curing her ulcer and surprisingly, after five months of treatment, and while the patient was about to undergo the standard test prior to the taking of the conventional treatment (tritherapy), it was discovered that not only the ulcer had been entirely cured but also that her CD4 level was equal to 635 units per mm³ of blood (knowing that before the treatment it was less than 500 units per mm³ of blood) and the viral load was almost undetectable, starting the conventional treatment becoming unnecessary.

The aforementioned patient is alive, following the taking of the composition according to the invention, and in good health in Cuba since June 2005 without any additional treatment of any kind.

### The Five Following Patients

Five other seropositive patients of Ukrainian origin were treated with the composition according to the invention.

Their seropositivity condition was detected in 1997 and in 1998; three of them were additionally affected by hepatitis C before following the treatment according to the invention, these five patients were closely followed by their attending physician.

A preliminary examination of their clinical health was carried out and all of them had symptoms of anxiety, fever, muscular pains, pains in the liver, fatigue, nauseas, vomiting, losses of appetite, and headaches. Various blood samples were analyzed by laboratories at various dates since the beginning of the treatment right up to 70 days after it. Three clinical and histological examinations were carried out, first upon arrival of the patients, one month after the treatment, and two months after the treatment.

After ten days of treatment, all the aforementioned symptoms suffered by the atients which were in the second phase of the disease, had disappeared.

All the patients were obviously in good health, no longer suffered from hepatitis C, had increased their body volume, had no fever, and noticed that their sexual libido, which was quasi-inexistent before the treatment, had reappeared.

Similar improvements were observed with the results of haematological tests. CD4 and viral load measurements were carried out at different steps of the treatment and a slight improvement in the CD4 level was observed. In an analogous way, the results relative to viral load were satisfactory.

The Last Three Patients

Additional clinical tests were carried out on three other patients in collaboration with a recognized international centre for research on the HIV, located in Paris.

Thus, at mid-August 2007, three HIV-bearing patients, natives of Lebanon, more specifically two men and one woman which had been infected by each other, were approached. Both men were experiencing the first stage of the disease, while the 26-year old woman had already been summoned by her attending physician for starting the conventional treatment (tritherapy).

The three patients decided to scrupulously follow the treatment method developed for the composition according to the invention upon recommendation from the aforementioned research centre, the woman having further refused the conventional treatment.

Before beginning the treatment with the composition according to the invention, a first blood sample was taken from the three patients (<<day 0>> of Tables 1 and 2). The results were passed on to the aforementioned research centre. All the three patients began to take the composition according to the invention on Aug. 19^{th} and 25^{th} 2007 by orally ingesting every day 20 grams of the composition, 3 to 4 times daily and at intervals of about six hours.

A second blood sample was taken on the 4^{th} of October of the year 2007 and was directly sent to the aforementioned research centre for analysis, evaluation and comparison with the results of previous tests.

The results were surprising since, as visible in Table No. 1, after 40 days, the viral load of all the patients had drastically decreased by 76% for the first patient, 88% for the second and 92% for the third.

Considering these positive results, the three patients continued to regularly take the composition according to the invention for a further 160 days, thereby bringing the total duration of the treatment up to 200 days, as mentioned in Tables 1 and 2.

The results obtained from the blood samples confirm the effectiveness of the composition over time:

For a same patient, for example patient No. 1, the lowering of the viral load is constant from the beginning to the end of the treatment and this viral load is lowered down to 95% at the very end of the treatment (200^{th} day).

As for the patient No. 2, a drastic lowering is also observed between the beginning of the treatment and the 40^{th} day of the treatment since the number of viral copies per mL of blood is lowered by 88% so that subsequently from the 40^{th} day, it is still located below 12,000 copies and ends with about 8,000 copies per mL of blood, which represents a 83% reduction of the viral load relatively to the beginning of the treatment.

The same applies for patient 3 with, as this is visible on the graph 2, a drastic lowering by 92% of the viral load during the first 40 days and then a stabilization below 20,000 copies per mL of blood between the 40^{th} and 200^{th} day of treatment, ending with a low level around 5,000 copies per mL of blood, which represents a lowering by 94% relative to the very beginning of the treatment.

As regards the change in the number of T lymphocytes per mm³ of blood, it is observed that the latter slightly increase between the beginning and the end of the treatment for patient No. 1 since a 17% increase of these lymphocytes is measured between day 0 and the 200^{th} day of the treatment. A much more significant increase is observed as regards patients Nos. 2 and 3 since between the beginning of the treatment and the end of the latter for patient No. 2, the number of T lymphocytes per mm³ of blood increased by 86%, and 78% for patient No. 3, with a remarkable increase at the end of the 144^{th} day by 102% since the lymphocytes of patient No. 3 attain 930 species per mm³ of blood at this instant.

It is important to note that all the patients mentioned above were and are still under medical supervision by specialist doctors and that this supervision confirms their good health. In addition, no harmful side effect was observed.

According to the investigations carried out, the effect of the composition according to the invention is due to its drastic action on the virus itself.

As the tests were carried out on the patients, the composition, the mode of administration and manufacturing method thereof were optimized.

From this optimization phase, it can be seen that GAFT and flower of sulfur are the basic elements for obtaining, when combined with each other, a reducing effect on the viral load and an increasing effect on the CD4s.

In GAFT, tannin agents and catechin are found (cf. paragraph <<GAFT>> of chapter <<Details on the main constitutive ingredients of the composition>>).

Also, in gambier (Uncaria Gambir), tannin agents and catechin are again found.

The gambier, containing tannin agents and catechin, may therefore form with flower of sulfur and instead of GAFT, the basic elements for obtaining, when combined with each other, an effect of reducing the viral load and increasing the CD4s.

Also, other plants containing catechin and tannin agents, such as those mentioned in the following part giving details on the ingredients used, may form with flower of sulfur the basic elements in order to obtain, when combined with each other, an effect of reducing the viral load and of increasing CD4s.

Nigella oil is also a constitutive element of the composition substantially involved in the effect of reducing the viral load and increasing the CD4s. The compositions containing the plants GAFT and gambier, in combination with nigella oil and flower of sulfur, prove to be the most satisfactory in terms of the effect of reducing the viral load and increasing the CD4s.

The composition according to the invention therefore proves to be effective in treating patients affected by the HIV virus, by hepatitis C and suffering from ulcers.

It is obtained from plants and minerals which prevents it from causing harmful side effects.

Its making method is further simple to realize.

Of course, none of the tests carried out was established by disclosing to anybody either the constitutive ingredients of the composition or the production method thereof. Indeed, the patients ingested the composition according to the invention immediately after having received it, in the presence of the medical team.

The details of the constitutive ingredients of the composition according to the invention are provided in the following.

### Details on the Main Constitutive Ingredients of the Composition:

### 1) GAFT or Common Agrimony or Agrimonia Eupatoria

### Scientific Name:

Agrimonia eupatoria L

### Family:

### Rosaceae

Other designations: common agrimony, church steeples, sticklewort, agrimony, cockleburr

### Botany and Geography

Origin: Hedges, woods and soils throughout Europe, North Africa, Western and Northern Asia.

Botanical description:—Perennial herbaceous plant with a height of 40-60 cm, with a reddish hairy stem—large leaves, divided in unequal segments—very numerous small yellow flowers grouped in long terminal clusters—Fruit: 1-2 achenes enclosed in a bristly calyx

### Parts Used:

### Leaves and Flowered Heads Constituents:

Tannins, coumarins, flavan-3-ol (notably formed by catechins), quercetin, kaempferols, luteolin, apigenin, different phenolic acids, vitamins B and K

### Risks for Humans:

Probably none

### 2) Gambier or Gambir or Uncaria Gambir

### Kingdom: Plantae

### Sub-kingdom: Tracheobionta

### Division: Magnoliophyta

### Class: Magnoliopsida

### Sub-class: Asteridae

### Order: Rubiales

### Family: Rubiaceae

### Genus: Uncaria

Binomial name: Uncaria gambir (W. Hunter) Roxb., 1824

### Order: Gentianales

### Use:

Gambier is a medicinal plant with tonic and astringent properties which is used for treating inter alia bums, diarrheas, coughs, sore throats and ulcers. The plant contains tannic acid and catechin.

2.1 Tannic acid

This acid may be obtained by degradation of tannins by microorganisms.

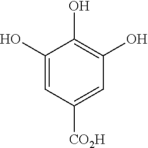

Tannins are complex combinations of glucose and gallic acid which are found in oak bark, chestnut bark, and in gall nuts. Tannins make skins rot-proof, whence their use in treating leather. They are also used for the production of ink.

### 2.2 Catechin:

Catechin is a molecule from the family of flavonoids of the sub-class of flavanols. It is also known as catechol.

Initially discovered in the fruit of cutch acacia (Acacia catechu) from which its name is derived, catechin and its numerous isomers are powerful antioxidants which help in preventing inflammatory and coronary diseases.
Catechin is a chiral molecule which has two symmetric forms: (+)-catechin and (−)-catechin. In order to avoid confusion between the class and the compound, the latter is designated in this context by (+/−)-catechin.

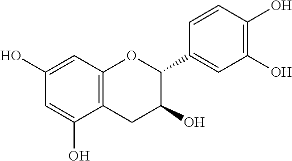

(+)-catechin is an antibiotic and an antioxidant which prevents formation of free radicals.

Plants other than gambier are rich in catechins and in tannin acids:
the tea shrub (Camellia sinensis): flavonoids from tea shrub leaves consist of 80% of flavanols, i.e. (+)-catechin C, (−)-epicatechin EC, (+)-gallocatechin GC and their gallic esters.
   Green, oolong and black teas are notable for the extent of enzymatic oxidation undergone by the leaves during a process incorrectly called <<fermentation>>. The flavonoids of green tea, like those of fresh leaves, consist of 80% of flavanols, but after oxidation, only 20-30% remain in black tea. Tea is known as being rich in tannin agents.
grape vine (Vitis vinifera): the grapes are rich in (+)-catechin, (−)-epicatechin and its gallic ester, epicatechin-3-gallate. These flavanols are concentrated in the seeds, at various doses depending on the vine variety, on the soil, on the vintage, on the phenolic maturity. High concentrations are found in the grape seeds of Merlot and Cabernet Sauvignon grapes. The grapes are rich in tannin agents.
the cocoa tree (Theobroma cacao): cocoa beans contain 12-18% of polyphenols (in % of dry materials) with about 35% of the latter in the form of (−)-epicatechin (for non-fermented Forastero beans). In order to become cocoa, the beans have to undergo fermentation, drying and roasting. During these numerous operations, the major part of the catechins and procyanidols are converted into quinones. In cocoa, flavanols however remain a majority among the polyphenols, with first (−)-epicatechin followed by (+)-catechin, (+)-gallocatechin, and (−)-epigallocatechin as well as proanthocyanidols consisting of 2 or 3 units of (+)-catechol and/or (−)-epicatechol, i.e. procyanidols B1, B2, B3, B4, B5, C1, and D. The high roasting temperatures convert parts of the (−)-epicatechin into its epimer (−)-catechin. Commercial cocoas from the Ivory Coast contain from 2.2 to 4.8 g/kg of epicatechin. Cocoa beans also contain tannin agents.
the beans are rich in catechins and in tannin agents
many kinds of fruit are also rich in catechins and tannin agents: firstly, blackberries, of course grapes, followed by cherries, apricots, raspberries, apples, plums, strawberries, pears, and peaches.

### 3) Flower of Sulfur

other designation: CREAM OF SULFUR
other designation: FLOWER OF SULFUR
note: 2179
salt or derivative: COLLOIDAL SULFUR
salt or derivative: SULFUR PRECIPITATE
salt or derivative: WASHED SUBLIMED SULFUR
Sulfur Chemical classes
Constituents: at least 99.5 percent of sulfur
4) Nigella Sativa (Habbat al Barakah or Nigella or black cumin vegetable oil)

### Composition

Myristic acid: 5%
Palmitic acid: 11.2-13.7%
Stearic acid: 2.1-2.6%
Palmitoleic acid: 0.1%
Oleic acid: 20.0-23.7%
Linoleic acid (omega 6): 57.9%
Alpha-linolenic acid (omega 3): 0.2%
Arachidic acid: 1.3%

Oil from Nigella (or Nigella or Nigella oil) is an oil of brown green color with a spicy odor.

The oil should be cold-extracted.

### 5) Menthol

Used in crystalline form

### 6) Ethanol

Used pure
7) Pure honey
The viscosity of the honey should be substantial in order to facilitate absorption of the substance by the stomach.

1. A composition for treating or preventing viral diseases of the blood comprising flower of sulfur, at least one plant containing tannin agents and catechin, and a pharmaceutically acceptable carrier, wherein the plant is selected from Agrimonia Eupatoria and Uncaria gambir.

2-3. (canceled)

4. The composition according to claim 1, including both Agrimonia Eupatoria and Uncaria gambir).

5. The composition according to claim 1 further comprising Nigella.

6. The composition according to claim 5, wherein the Nigella is present as an oil.

7. The composition according to claim 1 further comprising an antiputrid agent.

8. The composition according to claim 7, wherein the antiputrid agent is menthol.

9. The composition according to claim 1, further comprising a coating agent.

10. The composition according to claim 9, wherein the coating agent is honey.

11. A method for making a composition based on plants for treating or preventing viral diseases of the blood comprising mixing flower of sulfur, a pharmaceutically acceptable support, and at least one plant containing tannin agents and catechin and selected from Agrimonia Eupatoria and Uncaria gambir.

12. (canceled)

13. The method according to claim 11, including mixing both Agrimonia Eupatoria and Uncaria gambir with the flower of sulfur and the pharmaceutically acceptable support.

14. The method according to claim 11 including adding nigella oil.

15. The method according to claim 11, including obtaining the Agrimonia Eupatoria by distillation.

16. The method according to claim 11, wherein the Uncaria gambir is a powder.

17. The method according to claim 19 including dissolving the menthol, as menthol crystals, in pure alcohol before adding the menthol to the mixture.

18. The method according to claim 11, wherein the pharmaceutically acceptable carrier comprises honey.

19. The method according to claim 11 including adding menthol to the mixture as an antiputrid agent.
